(19) United States
(12) Patent Application Publication
(10) Pub. No.: US 2021/0210175 A1
(43) Pub. Date: Jul. 8, 2021

(54) SYSTEM AND METHOD FOR CROSS-AUTHENTICATION OF MEDICAL DATABASES FOR VERIFICATION OF ELECTRONIC HEALTH RECORDS INFORMATION

(71) Applicant: DAV ACQUISITION CORP., Orlando, FL (US)

(72) Inventors: Jennifer SETHRE, Austin, TX (US); Aquiles MATA, Lakeway, TX (US); Gustavo RINCON, Lakeway, TX (US); Michael A. LIBERTY, Windermere, FL (US)

(73) Assignee: DAV ACQUISITION CORP., Orlando, FL (US)

(21) Appl. No.: 16/888,143

(22) Filed: May 29, 2020

Related U.S. Application Data
(60) Provisional application No. 62/957,080, filed on Jan. 3, 2020.

(51) Int. Cl. G16H 10/60; G06F 16/23; G16H 70/40; G16H 50/20; G16H 15/00; G06F 21/62

(52) U.S. Cl. CPC G16H 10/60; G06F 16/2365; G06F 21/6245; G16H 50/20; G16H 15/00; G16H 70/40

(57) ABSTRACT

Systems and methods directed to reconciling electronic health records of a patient are disclosed. For example, a system includes: a memory storing instructions that implement an application for reconciling electronic health records of a patient; and a processing device communicatively coupled to the memory. The processing device capable of executing the application to: receive an indicator of permission to access an electronic health record of the patient maintained by a health provider; access, using the indicator, the electronic health record; determine if there are any inconsistencies or contraindications between the electronic health record and another electronic health record maintained by another health provider; and provide a notification of an inconsistency or a contraindication to the health provider and the other health provider.

### CROSS-REFERENCE TO RELATED APPLICATION(S)

This application claims priority to and the benefit of U.S. Provisional Application Patent Ser. No. 62/957,080, filed Jan. 3, 2020, the entire disclosures of which is hereby incorporated by reference.

### BACKGROUND

Patients face hurdles in authorizing data exchange of electronic health records (EHRs). Conventional technologies lack a common interface or standard system that orchestrates EHR access across databases of disparate health providers. The lack of communication and standardization between health providers in maintaining EHRs often cause inconsistencies or contraindications between the EHRs.

### SUMMARY

Representative embodiments set forth herein disclose various techniques for enabling a system and a method for verifying medical records across disparate health platforms.

In one embodiment, a system includes: a memory storing instructions that implement an application for reconciling electronic health records of a patient; and a processing device communicatively coupled to the memory. The processing device is capable of executing the application to: receive an indicator of permission to access an electronic health record of the patient maintained by a health provider; access, using the indicator, the electronic health record; determine if there are any inconsistencies or contraindications between the electronic health record and another electronic health record maintained by another health provider; and provide a notification of an inconsistency or a contraindication to the health provider and the other health provider.

In another embodiment, a tangible, non-transitory computer-readable medium stores instructions that, when executed, cause a processing device to execute the following steps disclosed. The steps include receiving an indicator of permission to access an electronic health record of the patient maintained by a health provider; accessing, using the indicator, the electronic health record; determining if there are any inconsistencies or contraindications between the electronic health record and another electronic health record maintained by another health provider; and providing a notification of an inconsistency or a contraindication to the health provider and the other health provider.

In still yet another embodiment, a method comprises: receiving an indicator of permission to access an electronic health record of the patient maintained by a health provider; accessing, using the indicator, the electronic health record; determining if there are any inconsistencies or contraindications between the electronic health record and another electronic health record maintained by another health provider; and providing a notification of an inconsistency or a contraindication to the health provider and the other health provider.

### BRIEF DESCRIPTION OF THE DRAWINGS

For a detailed description of example embodiments, reference will now be made to the accompanying drawings in which:

FIG. 1 illustrates a block diagram of an example of a cloud services network that enables accessing of electronic health records (EHRs) maintained by disparate health providers, in accordance with various embodiments.

FIG. 2 illustrates block diagram of exemplary embodiment of EHR reconciliation application, in accordance with various embodiments.

FIG. 3 depicts a method for reconciling EHRs of a patient, in accordance with various embodiments.

FIGS. 4 and 5 illustrate example notifications received by health providers, in accordance with various embodiments.

FIG. 6 illustrates a detailed view of a computing device that can be used to implement the various components described herein, in accordance with various embodiments.

### NOTATION AND NOMENCLATURE

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Patients face hurdles in authorizing data exchange of electronic health records (EHRs). Conventional technologies lack a common interface or standard system that orchestrates EHR access across databases of disparate health providers. The lack of communication and standardization between health providers in maintaining EHRs often causes inconsistencies or contraindications between the EHRs.

In one embodiment, a system includes: a memory storing instructions that implement an application for reconciling electronic health records of a patient; and a processing device communicatively coupled to the memory. The processing device capable of executing the application to: receive an indicator of permission to access an electronic health record of the patient maintained by a health provider; access, using the indicator, the electronic health record; determine if there are any inconsistencies or contraindications between the electronic health record and another electronic health record maintained by another health provider; and provide a notification of an inconsistency or a contraindication to the health provider and the other health provider.

In another embodiment, a tangible, non-transitory computer-readable medium storing instructions that, when executed, cause a processing device to execute the following steps disclosed. The steps include receiving an indicator of permission to access an electronic health record of the patient maintained by a health provider; accessing, using the indicator, the electronic health record; determining if there are any inconsistencies or contraindications between the electronic health record and another electronic health record maintained by another health provider; and providing a notification of an inconsistency or a contraindication to the health provider and the other health provider.

In still yet another embodiment, a method comprises: receiving an indicator of permission to access an electronic health record of the patient maintained by a health provider; accessing, using the indicator, the electronic health record; determining if there are any inconsistencies or contraindications between the electronic health record and another electronic health record maintained by another health provider; and providing a notification of an inconsistency or a contraindication to the health provider and the other health provider.

An EHR as used herein refers to a digital version of health records associated with a patient. EHRs include medical and treatment histories of patients but can go beyond standard clinical data collected by a doctor's office/health provider. For example, EHRs may include a patient's medical history, diagnoses, medications, treatment plans, immunization dates, allergies, radiology images, and laboratory and test results. A health provider as used herein refers to entities that provide health services to patients such as (but not limited to) hospitals, doctor offices, laboratories, specialists, medical imaging facilities, pharmacies, emergency facilities, and school and workplace clinics.

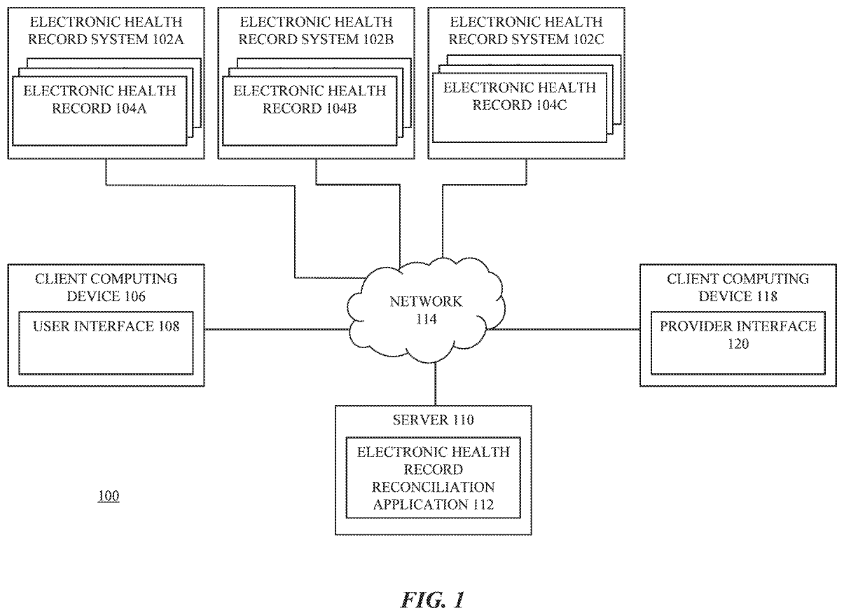

FIG. 1 is a block diagram of an example of a cloud services network 100 that enables accessing of EHRs maintained by disparate health providers. As shown in FIG. 1, cloud services network 100 includes a client computing device 106, a client computing device 118, a server 110, and EHR systems 102A, 102B, and 102C. As further shown in FIG. 1, server 110 hosts an EHR reconciliation application 112 and EHR systems 102A, 102B, and 102C respectively include EHR resources 104A, 104B, and 104C.

EHR systems 102A, 102B, and 102C represent network-connected, enterprise-wide information systems or other information networks of a health provider. In FIG. 1, EHR systems 102A, 102B, and 102C are each associated with a different, disparate health provider. For illustration purposes, cloud services network 100 is shown to include EHR systems 102A, 102B, and 102C. However, cloud services network 100 may include any number of EHR systems. Additionally, in accordance with embodiments described herein, EHR systems 102A, 102B, and 102C may include any number of EHR resources associated with one or more patients.

Client computing devices 106 and 118 may be any type of stationary or mobile computing device, including a mobile computer or mobile computing device (e.g., a smart phone, a laptop computer, a notebook computer, a tablet computer such as an Apple iPad™, a netbook, etc.), a wearable computing device (e.g., a smart watch, a head-mounted device including smart glasses such as Google® Glass™, etc.), or a stationary computing device such as a desktop computer or PC (personal computer). Server 110 may comprise one or more server devices and/or other computing devices. EHR systems 102A, 102B, and 102C may also comprise one or more server devices and/or other computing devices. EHR systems 102A, 102B, and 102C may also comprise one or more data storage devices or systems. Example data storage devices include but are not limited to a magnetic disc (e.g., in a hard disk drive), an optical disc (e.g., in an optical disk drive), a magnetic tape (e.g., in a tape drive), a RAM device, a ROM device, network-attached storage, or the like. Example data storage systems include but are not limited to a storage area network. In FIG. 1, EHR resources 104A, 104B, and 104C may be respectively stored in one or more data storage devices or systems of EHR systems 102A, 102B, and 102C.

In FIG. 1, components of cloud services network 100 including client computing device 106, client computing device 118, server 110, and EHR systems 102A, 102B, and 102C may be communicatively connected via a network 114. Network 114 may include one or more networks. These one or more networks may include, for example, a local area network (LAN), a wide area network (WAN), a personal area network (PAN), and/or a combination of communication networks, such as the Internet.

EHR reconciliation application 112 may be a cloud-based or server-based computer program that is integrated into Internet-connected computing devices and/or computer programs of the computing devices. For example, a user (such as a patient) of client computing device 106 may interact with EHR reconciliation application 112 through a user interface 108 executing on client computing device 106. As another example, a user (such as a health provider professional including doctors, nurses, physical therapists, pharmacist, etc.,) of client computing device 118 may interact with EHR reconciliation application 112 through a provider interface 120 executing on client computing device 118.

In some embodiments, user interface 108, provider interface 120, and EHR reconciliation application 112 are example components of a cloud application hosted in cloud services network 100, where user interface 108 and provider interface 120 are front-end components of the cloud application and EHR reconciliation application 112 is a back-end component of the cloud application. For example, user interface 108 and provider interface 120 may be represented as a web page displayed in a web browser. As another example, user interface 108 and provider interface 120 may be an Internet-enabled application executing on client computing device 106 and client computing device 118, respectively. As such, in accordance with embodiments described herein, a user interface (such as user interface 108 and provider interface 120) of EHR reconciliation application 112 may be implemented in one or more end user computing devices (such as client computing devices 106 and 118), and EHR reconciliation application 112 may be implemented on one or more servers that are accessible to the one or more end user computing devices via one or more networks (such as network 114). Still other implementations of user interface 108 and provider interface 120 are possible.

EHR reconciliation application 112 is configured to access a EHRs of a patient that are maintained by disparate health providers. In some embodiments, a patient using client computing device 106 may interact with user interface 108 (e.g., through utterances of one or more words, typing of a request, or uploading of an image), and user interface 108 may capture user input representing a request of the patient from the interaction and provide the user input to EHR reconciliation application 112. For example, in FIG. 1, a user of client computing device 106 may request access, via user interface 108, to EHRs of the user from EHR systems 102A, 102B, and 102C. In some embodiments, EHR reconciliation application 112 may interpret the user input and assist the patient with the request, such as retrieving specific EHR resources and/or EHR resources (such as EHR resources 104A, 104B, and 104C) from particular periods of time, by invoking a service of a third-party EHR system and interacting therewith to fulfill the request. The request may be performed through services accessible by EHR reconciliation application 112 through application programming interfaces (APIs) exposed by those services provided by EHR systems 102A, 102B, and 102C. As used herein, the term “service” broadly refers to a computer program that can be accessed by other computer programs to perform one or more functions. One non-limiting example of a service is a Web service.

Further, in accordance with embodiments described herein, EHR systems 102A, 102B, and 102C may include one or more services. The one or more services may expose an API that enables EHR reconciliation application 112 and other computer programs to interact therewith via network 114. The services may comprise any type of network-accessible service such as a patient EHR retrieval service, a provider-patient messaging service, and an appointment reminder and scheduling service. Similarly, a health provider professional using the provider interface 120 may, via user interface 108, request EHR reconciliation application 112 to access EHRs of one or more patients maintained by disparate health providers. For example, a professional employed by a health provider associated with EHR system 102A may access, via provider interface 120, EHRs of EHR systems 102B and 102C.

In some embodiments, before accessing EHR resources on behalf of a patient, EHR reconciliation application 112 may need to request authorization from an EHR system to access EHR resources of the EHR system. For example, in FIG. 1, EHR reconciliation application 112 may obtain (e.g., via OAuth 2.0 authorization framework) authorization to access EHR resource 104A from EHR system 102A (e.g., via one or more authorization servers of EHR system 102A) on behalf of the user of client computing device 106. More specifically, EHR reconciliation application 112 may send a request for authorization (e.g., a Hypertext Transfer Protocol (HTTP) message) to EHR system 102A to access EHR resource 104A, which is an EHR of the user. The request may include a unique identifier associated with EHR reconciliation application 112, and based on the unique identifier, EHR system 102A may decide to grant or deny access to EHR resource 104A. In some embodiments, EHR reconciliation application 112 may need to register with EHR system 102A before being able to interact with EHR system 102A. During the registration process, EHR system 102A may issue EHR reconciliation application 112 an identifier (e.g., a unique string representing registration information provided by EHR reconciliation application 112). A patient or an agent of the patient may initiate the registration of EHR reconciliation application 112 with EHR systems via user interface 108.

Continuing with the example described above, EHR system 102A may communicate a decision to grant or deny access to EHR resource 104A to EHR reconciliation application 112 by returning an authorization code (or, if denying access, an error response). EHR reconciliation application 112 may exchange, with EHR system 102A, the authorization code for a token (e.g., an access token). Using the token, EHR reconciliation application 112 may access EHR resource 104A (e.g., from a resource server of EHR system 102A that serves EHR resource 104A). In some embodiments, EHR system 102A may decide to grant or deny access based on additional parameters or information included in the authorization request from EHR reconciliation application 112. For example, EHR system 102A may deny access based on information included in the authorization request about a system configuration or a security misconfiguration of client computing device 106.

Additionally, or alternatively, EHR system 102A may require end-user authorization through user interface 108. For example, a user of the client computing device 106 may be prompted to enter user credentials (e.g., a username and password) via user interface 108. EHR system 102A may validate the user credentials and provide an indicator of permission to access (e.g., an access token, refresh token, etc.) EHR resource 104A to EHR reconciliation application 112. EHR reconciliation application 112 may use the indicator to access EHR resource 104A of EHR system 102A. Still yet, in some embodiments, the request for authorization may include personal identifying information associated with the user and/or a patient (e.g., a patient ID, social security number, etc.) that EHR system 102A may need to validate.

More specifically, in some embodiments, EHR reconciliation application 112 may provide the indicator of permission to access EHR resource 104A to a resource server of EHR system 102A. For example, the resource server may validate the indicator of permission and ensure that the indicator has not expired and that the scope of the indicator covers the requested resource. The resource server may also validate other parameters included in the indicator. For example, the scope of access to an EHR resource included in the indicator may be based on user-level scopes that dictate specific types of EHR data that a user can access. In FIG. 1, with a valid indicator of permission to access EHR resource 104A, EHR reconciliation application 112 may access EHR resource 104 by issuing an API call (e.g., a RESTful API) to an endpoint on the resource server of EHR system 102A. The resource server identifies which of the EHR resources it serves satisfies the request and returns the results including the identified EHR resources (EHR resource 104A) in a message to EHR reconciliation application 112. Furthermore, in some embodiments, EHR reconciliation application 112 may provide the resource server of EHR system 102A with one or more parameters indicating the type of EHR resource that the resource server should provide. For example, the one or more parameters may indicate a categorical grouping of EHR data (e.g., allergies, medicines, appointments, family history, procedures, immunizations, medical tests, etc.). Additionally, the one or more parameters may specify a specific date and time and/or a period of time that the EHR resource was created and/or keywords that the EHR resource may include.

Moreover, EHR system 102A may be further configured to enforce access rules and policies set by the health provider associated with EHR system 102A. For example, EHR system 102A may determine based on an access policy what EHR resources the user and/or EHR reconciliation application 112 can access and interact with. An access policy may outline which users or groups of users' and/or what applications' network cloud traffic should be able to access EHR resources of EHR system 102A. In embodiments, an information technology (IT) administrator for the health provider associated with EHR system 102A may set access policies for applications (e.g., EHR reconciliation application 112) and/or users of client devices (e.g., client computing devices 106 and 118) that may want to access EHR resources of EHR system 102A. For example, EHR system 102A may evaluate a user's login (e.g., username and password) to determine if there is a policy associated with that user. Say for example, the user is a patient of the health provider associated with EHR system 102A and the health provider has a policy that patients are not permitted to access notes of its professionals. EHR system 102A may prevent the user from accessing any EHR resources including notes of health provider professionals. EHR reconciliation application 112 may receive EHR resources 104B and 104C from EHR systems 102B and 102C in a similar manner as described previously with reference to EHR reconciliation application 112 receiving EHR resource 104A from EHR system 102A.

After receiving EHR resources associated with a patient, EHR reconciliation application 112 is configured to determine if there are any inconsistencies or contraindications between the EHR resources. Inconsistencies as referenced herein refers to conflicts between EHR resources. For example, an EHR resource of an EHR system may indicate a patient had a left kidney removed during a pervious surgery, whereas an EHR resource of another EHR system may indicate that a right kidney was removed during the previous surgery. Contraindications as referenced herein refers to situations in which a treatment or recommendation of a health care provider may be harmful to a patient. In medicine, a relative contraindication refers to situations in which the administration of two drugs together or the performance of two procedures together may be harmful to the patient and absolute contraindication refers to an event or substance that could cause a life-threatening situation for a patient. For example, some treatments may cause unwanted or dangerous reactions in people with allergies, high blood pressure, or pregnancy. Additionally, many medicines should not be used together by the same person.

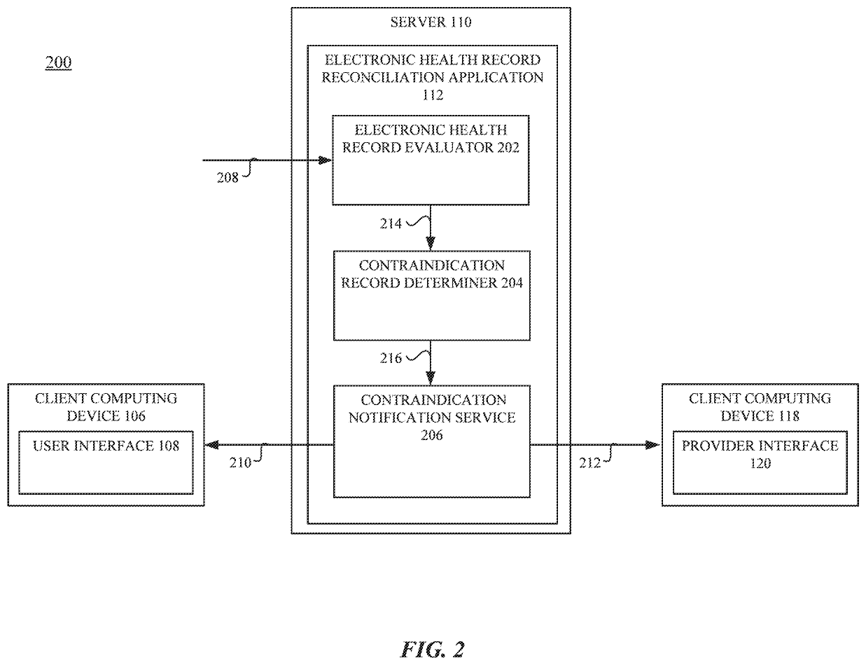

FIG. 2 provides a more detailed block diagram of another exemplary embodiment of EHR reconciliation application 112 in FIG. 1. As shown in FIG. 2, a system 200 comprises EHR reconciliation application 112, which includes an EHR evaluator 202, an inconsistency/contraindication determiner 204, and an inconsistency/contraindication notification service 206. EHR evaluator 202 is configured to receive EHR resources 208 (e.g., EHR resources 104A, 104B, and 104C in FIG. 1) and process EHR resources 208. As described, EHR resources 208 associated with the user may be received from disparate EHR systems. Often disparate EHR systems maintain EHR resources using different data content storage standards. As such, EHR evaluator 202 may need to standardized EHR resources 208 so that EHR resources 208 are structured and comparable.

EHR evaluator 202 may also use natural language processing (NLP) and other artificial intelligence (AI) tools to process and categorize EHR resources 208. For example, EHR evaluator 202 may use NLP to extract and interpret hand written notes and text from EHR resources 208. As another example, EHR evaluator 202 may use imaging extraction techniques, such as optical character recognition (OCR) and/or use a machine learning model trained to identify and extract certain information from EHR resources 208. OCR refers to electronic conversion of an image of printed text into machine-encoded text and may be used to digitize information in EHR resources 208. As another example, pattern recognition and/or computer vision may also be used to extract information from EHR resources 208. Computer vision may involve image understanding by processing symbolic information from image data using models constructed with the aid of geometry, physics, statistics, and/or learning theory. Pattern recognition may refer to electronic discovery of regularities in data through the use of computer algorithms and with the use of these regularities to take actions such as classifying the data into different categories and/or determining what the symbols represent in the image (e.g., words, sentences, names, numbers, identifiers, etc.).

Finally, natural language understanding (NLU) may be performed on EHR resources 208. NLU techniques may process unstructured data using text analytics to extract entities, relationships, keywords, semantic roles, and so forth. Still yet in some embodiments, EHR evaluator 202 may store processed and/or unprocessed received EHR resources 208 locally or externally in a central repository.

Inconsistency/contraindication determiner 204 is configured to receive processed and/or unprocessed EHR resources 214 (including EHR resources 208) from EHR evaluator 202 and to determine if there are any inconsistencies or contraindications between EHR resources 214. Additionally, or alternatively, inconsistency/contraindication determiner 204 may retrieve EHR resources 214 from a data store that EHR evaluator 202 stored EHR resources 214.

Assume for illustration purposes EHR resources 214 include any health records related to a medication history (e.g., including prescriptions, non-prescriptions, vitamins, herbals, and dietary supplements) of the user of client computing device 106. As described, in some embodiments, the user may request EHR evaluator 202 to access EHR resources related to a medication history of the user one or more EHR systems (e.g., EHR systems 102A, 102B, and 102C in FIG. 1). Additionally, or alternatively, EHR evaluator 202 may access EHR resources of the user automatically and/or periodically (e.g., monthly, yearly, etc.).

After receiving EHR resources 214, inconsistency/contraindication determiner 204 may compare drug interaction warnings issued by drug manufacturers and/or findings of drug trials or studies to medicines indicated in EHR resources 214. For instance, the user may be prescribed to take warfarin, a blood thinner, by a first health provider and recommended by second health provider to take aspirin, which is also a blood thinner. Using warfarin and aspirin simultaneously is an example of a relative contraindication. In some embodiments, inconsistency/contraindication determiner 204 may obtain published drug interaction warnings or findings of drug trials or studies by searching the Internet.

Furthermore, inconsistency/contraindication determiner 204 may use the AI tools described previously to determine if there are any inconsistencies or contraindications in EHR resources 214. For example, with continued reference to the example described above, inconsistency/contraindication determiner 204 may use one or more machine learning models for identifying potentially adverse relationships between medicines/drugs and conditions or characteristics of the patient indicated in EHR resources 214. For instance, some medicines may cause unwanted or dangerous reactions in patients with allergies, high blood pressure, or pregnancy.

The one or more machine learning models may be generated by a training engine and may be implemented in computer instructions that are executable by one or more processing devices of the training engine, server 110, the client computing device 106 and/or the client computing device 118. To generate the one or more machine learning models, the training engine may train, test, and validate the one or more machine learning models. The one or more machine learning models may refer to model artifacts that are created by the training engine using training data (e.g., EHR data, clinical trials data, patient-generated data, etc.) that includes training inputs and corresponding target outputs (e.g., adverse relationships between patient information indicated in EHR resources). The training engine may find patterns in the training data that map the training input to the target output and generate the machine learning models that capture these patterns. In addition, inconsistency/contraindication determiner 204 may use the one or more machine learning models to identify and present appropriate dose recommendations based on patient-specific conditions and characteristics indicated in the EHR resources. In some embodiments, although not depicted in FIG. 2, the one or more machine learning models and the training engine may be components of EHR reconciliation application 112.

After determining there is an inconsistency or a contraindication in EHR resources 214, inconsistency/contraindication determiner 204 may provide an indication 216 of the inconsistency or the contraindication to inconsistency/contraindication notification service 206. Inconsistency/contraindication notification service 206 may generate a notification, based on the indication 216, to inform the user of the inconsistency or the contraindication. For example, in FIG. 2, inconsistency/contraindication notification service 206 may generate a message 210 and provide message 210 to user interface 108 for display, thereby informing the user of the inconsistency or the contraindication.

In addition, in FIG. 2, inconsistency/contraindication notification service 206 may generate a message 212 and provide message 212 to provider interface 120 for display, thereby informing a health provider professional of the inconsistency or the contraindication. In some embodiments, any health provider maintaining an EHR resource involving the inconsistency or the contraindication may receive a notification of the inconsistency or the contraindication. Still yet in some embodiments, a health provider may receive a notification but the notification reveals limited information about the inconsistency or the contraindication. Health providers may be provided information related to the inconsistency or the contraindication on a “need-to-know” basis.

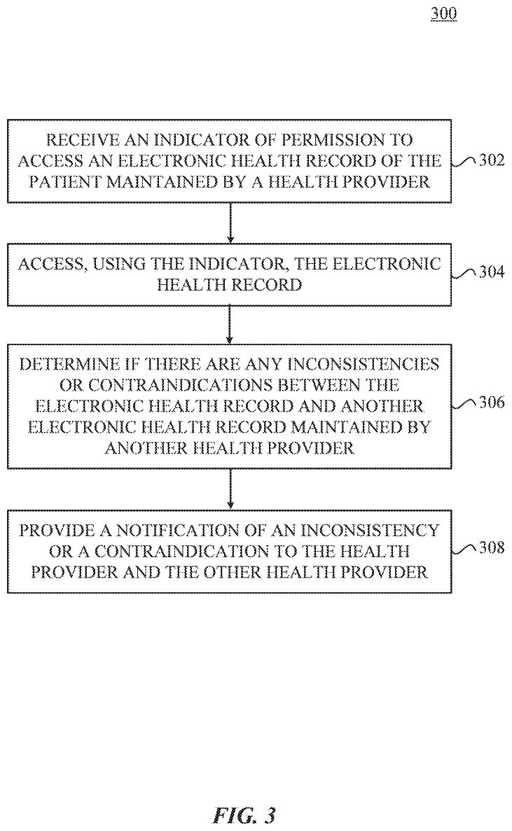

FIG. 3 will now be described to help further explain the foregoing. FIG. 3 illustrates a method 300 for reconciling electronic health records of a patient. As shown in FIG. 3, method 300 starts at step 302. At step 302, an indicator is received, where the indicator specifies permission to access an electronic health record of the patient maintained by a health provider. For example, with continued reference to FIGS. 1 and 2, EHR evaluator 202 receives an indicator of permission to access EHR resources 104A, 104B, and 104C from each of EHR systems 102A, 102B, and 102C.

At step 304, the electronic health record is accessed using the indicator. For example, with continued reference to FIG. 1 and FIG. 2, EHR evaluator 202 accesses, using the indicators received in step 302, to access EHR resources 104A, 104B, and 104C from EHR systems 102A, 102B, and 102C, respectively.

At step 306, it is determined if there are any inconsistencies or contraindications between the electronic health record and another electronic health record maintained by another health provider. For example, with continued reference to FIG. 1 and FIG. 2, inconsistency/contraindication determiner 204 determines if there are any inconsistencies or contraindications between EHR resources 104A, 104B, and 104C. For example, EHR resource 104A may indicate that the user was prescribed to take warfarin, a blood thinner, and EHR resource 104B may indicate that the user was recommended to take aspirin, which is also a blood thinner. Using warfarin and aspirin simultaneously is an example of a relative contraindication.

At step 308, a notification is provided of an inconsistency or a contraindication to the health provider and the other health provider. For example, with continued reference to FIG. 1 and FIG. 2, inconsistency/contraindication notification service 206 provides a notification of an inconsistency or a contraindication to an employee of the health provider associated with EHR system 102A via provider interface 120. Additionally, inconsistency/contraindication notification service 206 provides the notification of the inconsistency or the contraindication to an employee of the health provider associated with EHR system 102B via another provider interface.

Further, in some embodiments, a first health provider may be provided the notification of the inconsistency or the contraindication and another health provider may be provided a warning related to the inconsistency or the contraindication that does not disclose the inconsistency or the contraindication. In other words, the notification may be tailored to the health provider. For example, if two medicines prescribed to a patient by different health providers are determined to potentially have an adverse impact on the patient if taken together, the prescribing health providers may be notified of the contraindication. Say also the patient's sodium intake levels can impact the patient adversely when taking either of these two medicines. A nutritionist of the patient may only be warned to keep the patient's sodium intake under a certain level and not informed of the two medicines that the patient has been prescribed.

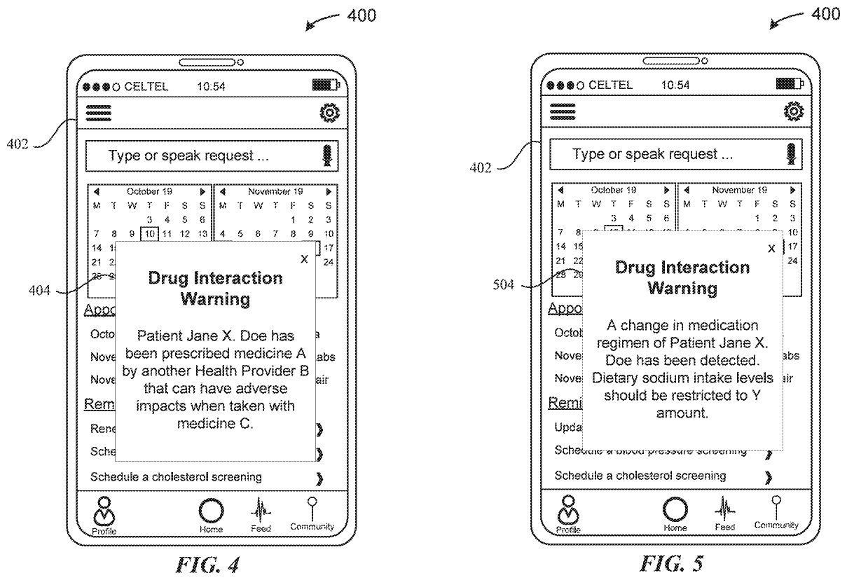

FIG. 4 provides an example notification 404 that might be displayed in an instance of a provider interface 402 (e.g., provider interface 120 in FIG. 1) for a professional of the health provider prescribing either of the medicines. As shown in FIG. 4, notification 404 recites: “Patient Jane X. Doe has been prescribed medicine A by another Health Provider B that can have adverse impacts when taken with medicine C.” In some embodiments, the provider interface may enable the two prescribing health providers to communicate to rectify or resolve the inconsistency or the contraindication.

FIG. 5 provides an example notification 504 that might be displayed in an instance of provider interface 402 (e.g., provider interface 120 in FIG. 1) for a nutritionist of a health provider. As shown in FIG. 5, notification 504 recites: “A change in medication regimen of Patient Jane X. Doe has been detected. Dietary sodium intake levels should be restricted to Y amount.”

Additionally, in some embodiments a patient may provide (via user interface 108 in FIG. 1) a list of health providers that the patient permits receiving notifications from inconsistency/contraindication notification service 206. In some embodiments, inconsistency/contraindication notification service 206 may determine which health providers of the list may receive a notification of the inconsistency or the contraindication. For example, inconsistency/contraindication notification service 206 may filter health providers based on a categorical grouping of the inconsistency or the contraindication. For instance, a physical therapy health provider may not need to receive notifications of a pharma chemical related inconsistency or contraindication.

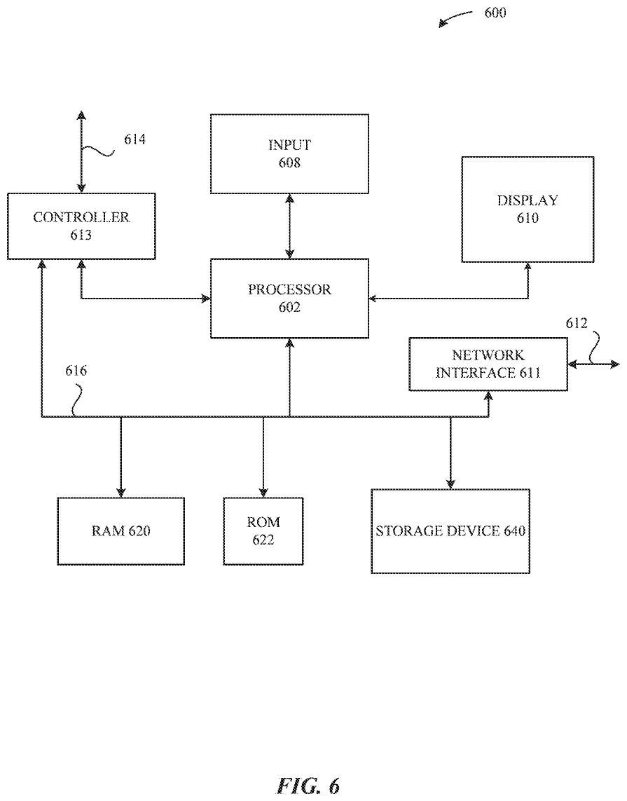

FIG. 6 illustrates a detailed view of a computing device 600 that can be used to implement the various components described herein, according to some embodiments. In particular, the detailed view illustrates various components that can be included client computing device 106, client computing device 118, server 110, and EHR systems 102A, 102B, and 102C illustrated in FIG. 1 and FIG. 2. As shown in FIG. 6, the computing device 600 can include a processor 602 that represents a microprocessor or controller for controlling the overall operation of the computing device 600. The computing device 600 can also include a user input device 608 that allows a user of the computing device 600 to interact with the computing device 600. For example, the user input device 608 can take a variety of forms, such as a button, keypad, dial, touch screen, audio input interface, visual/image capture input interface, input in the form of sensor data, and so on. Still further, the computing device 600 can include a display 610 that can be controlled by the processor 602 to display information to the user. A data bus 616 can facilitate data transfer between at least a storage device 640, the processor 602, and a controller 613. The controller 613 can be used to interface with and control different equipment through an equipment control bus 606. The computing device 600 can also include a network/bus interface 611 that couples to a data link 612. In the case of a wireless connection, the network/bus interface 611 can include a wireless transceiver.

As noted above, the computing device 600 also includes the storage device 640, which can comprise a single disk or a collection of disks (e.g., hard drives), and includes a storage management module that manages one or more partitions within the storage device 640. In some embodiments, storage device 640 can include flash memory, semiconductor (solid-state) memory or the like. The computing device 600 can also include a Random-Access Memory (RAM) 620 and a Read-Only Memory (ROM) 622. The ROM 622 can store programs, utilities or processes to be executed in a non-volatile manner. The RAM 620 can provide volatile data storage, and stores instructions related to the operation of processes and applications executing on the computing device.

The various aspects, embodiments, implementations or features of the described embodiments can be used separately or in any combination. Various aspects of the described embodiments can be implemented by software, hardware or a combination of hardware and software. The described embodiments can also be embodied as computer readable code on a computer readable medium. The computer readable medium is any data storage device that can store data which can thereafter be read by a computer system. Examples of the computer readable medium include read-only memory, random-access memory, CD-ROMs, DVDs, magnetic tape, hard disk drives, solid-state drives, and optical data storage devices. The computer readable medium can also be distributed over network-coupled computer systems so that the computer readable code is stored and executed in a distributed fashion.

Consistent with the above disclosure, the examples of systems and method enumerated in the following clauses are specifically contemplated and are intended as a non-limiting set of examples.

In an embodiment, a system, comprises: a memory storing instructions that implement an application for reconciling electronic health records of a patient; and a processing device communicatively coupled to the memory, the processing device capable of executing the application to: receive an indicator of permission to access an electronic health record of the patient maintained by a health provider; access, using the indicator, the electronic health record; determine if there are any inconsistencies or contraindications between the electronic health record and another electronic health record maintained by another health provider; and provide a notification of an inconsistency or a contraindication to the health provider and the other health provider.

In the embodiment of the foregoing system, the inconsistency or the contraindication is a pharma chemical contraindication.

In the embodiment of the foregoing system, the processing device is further capable of executing the application to: standardize the electronic health record; and store the standardized electronic health record in a central repository.

In the embodiment of the foregoing system, the processing device is further capable of executing the application to: provide the notification of the inconsistency or the contraindication to the patient.

In the embodiment of the foregoing system, the health provider is provided the notification of the inconsistency or the contraindication and the other health provider is provided a warning related to the inconsistency or the contraindication that does not disclose the inconsistency or the contraindication.

In the embodiment of the foregoing system, the processing device is further capable of executing the application to: receive a list of health providers that the patient permits receiving the notification of the inconsistency or the contraindication.

In the embodiment of the foregoing system, the processing device is further capable of executing the application to: determine a portion of the list of health providers to be provided the notification of the inconsistency or the contraindication based on a categorical grouping of the inconsistency or the contraindication.

In another embodiment, a method comprises: receiving an indicator of permission to access an electronic health record of the patient maintained by a health provider; accessing, using the indicator, the electronic health record; determining if there are any inconsistencies or contraindications between the electronic health record and another electronic health record maintained by another health provider; and providing a notification of an inconsistency or a contraindication to the health provider and the other health provider.

In the embodiment of the foregoing method, wherein the inconsistency or the contraindication is a pharma chemical contraindication.

In the embodiment of the foregoing method, the method further comprises: standardizing the electronic health record; and storing the standardized electronic health record in a central repository.

In the embodiment of the foregoing method, the method further comprises: providing the notification of the inconsistency or the contraindication to the patient.

n the embodiment of the foregoing method, the health provider is provided the notification of the inconsistency or the contraindication and the other health provider is provided a warning related to the inconsistency or the contraindication that does not disclose the inconsistency or the contraindication.

In the embodiment of the foregoing method, the method further comprises receiving a list of health providers that the patient permits receiving the notification of the inconsistency or the contraindication.

In the embodiment of the foregoing method, the method further comprises determining a portion of the list of health providers to be provided the notification of the inconsistency or the contraindication based on a categorical grouping of the inconsistency or the contraindication.

In another embodiment, a tangible, non-transitory computer-readable medium storing instructions that, when executed, cause a processing device to: receive an indicator of permission to access an electronic health record of the patient maintained by a health provider; access, using the indicator, the electronic health record; determine if there are any inconsistencies or contraindications between the electronic health record and another electronic health record maintained by another health provider; and provide a notification of an inconsistency or a contraindication to the health provider and the other health provider.

In the embodiment of the foregoing computer-readable medium, the inconsistency or the contraindication is a pharma chemical contraindication.

In the embodiment of the foregoing computer-readable medium, the processing device is further caused to: standardize the electronic health record; and store the standardized electronic health record in a central repository.

In the embodiment of the foregoing computer-readable medium, the processing device is further caused to: provide the notification of the inconsistency or the contraindication to the patient.

In the embodiment of the foregoing computer-readable medium, the health provider is provided the notification of the inconsistency or the contraindication and the other health provider is provided a warning related to the inconsistency or the contraindication that does not disclose the inconsistency or the contraindication.

In the embodiment of the foregoing computer-readable medium, the processing device is further caused to: receive a list of health providers that the patient permits receiving the notification of the inconsistency or the contraindication.

The foregoing description, for purposes of explanation, used specific nomenclature to provide a thorough understanding of the described embodiments. However, it should be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. A system, comprising:
a memory storing instructions that implement an application for reconciling electronic health records of a patient; and
a processing device communicatively coupled to the memory, the processing device capable of executing the application to:
generate one or more machine learning models trained to identify adverse relationships between information included in electronic health records;
receive an indicator of permission to access a first electronic health record of the patient maintained by a first health provider;
access, using the indicator, the first electronic health record;
determine a contraindication between the first electronic health record and a second electronic health record maintained by a second health provider using the one or more machine learning models to identify an adverse relationship between information included in the first electronic health record and the second electronic health record; and
provide a notification of the contraindication to the first health provider and the second health provider.

2. The system of claim 1, wherein the contraindication is a pharma chemical contraindication.

3. The system of claim 1, wherein the processing device is further capable of executing the application to:
standardize the first electronic health record; and
store the standardized electronic health record in a central repository.

4. The system of claim 1, wherein the processing device is further capable of executing the application to:
provide the notification of the contraindication to the patient.

5. The system of claim 1, wherein the first health provider is provided the notification of the contraindication and the second health provider is provided a warning related to the contraindication that does not disclose the contraindication.

6. The system of claim 1, wherein the processing device is further capable of executing the application to:
receive a list of health providers that the patient permits receiving the notification of the contraindication.

7. The system of claim 6, wherein the processing device is further capable of executing the application to:
determine a portion of the list of health providers to be provided the notification of the contraindication based on a categorical grouping of the contraindication.

8. A method, comprising:
generating one or more machine learning models trained to identify adverse relationships between information included in electronic health records;
receiving an indicator of permission to access a first electronic health record of a patient maintained by a first health provider;
accessing, using the indicator, the first electronic health record;
determining a contradiction between the first electronic health record and a second electronic health record maintained by a second health provider using the one or more machine learning models to identify an adverse relationship between information included in the first electronic health record and the second electronic health record; and
providing a notification of the contraindication to the first health provider and the second health provider.

9. The method of claim 8, wherein the contraindication is a pharma chemical contraindication.

10. The method of claim 8, further comprising:
standardizing the first electronic health record; and
storing the standardized electronic health record in a central repository.

11. The method of claim 8, further comprising:
providing the notification of the contraindication to the patient.

12. The method of claim 8, wherein the first health provider is provided the notification of the contraindication and the second health provider is provided a warning related to the contraindication that does not disclose the contraindication.

13. The method of claim 8, further comprising:
receiving a list of health providers that the patient permits receiving the notification of the contraindication.

14. The method of claim 13, the method further comprising:
determining a portion of the list of health providers to be provided the notification of the contraindication based on a categorical grouping of the contraindication.

15. A tangible, non-transitory computer-readable medium storing instructions that, when executed, cause a processing device to:
generate one or more machine learning models trained to identify adverse relationships between information included in electronic health records;
receive an indicator of permission to access a first electronic health record of a patient maintained by a first health provider;
access, using the indicator, the first electronic health record;
determine a contraindication between the first electronic health record and a second electronic health record maintained by a second health provider using the one or more machine learning models to identify an adverse relationship between information included in the first electronic health record and the second electronic health record; and
provide a notification of the contraindication to the first health provider and the second provider.

16. The computer-readable medium of claim 15, wherein the contraindication is a pharma chemical contraindication.

17. The computer-readable medium of claim 15, wherein the processing device is further caused to:
standardize the first electronic health record; and
store the standardized electronic health record in a central repository.

18. The computer-readable medium of claim 15, wherein the processing device is further caused to:
provide the notification of the contraindication to the patient.

19. The computer-readable medium of claim 15, wherein the first health provider is provided the notification of the contraindication and the second health provider is provided a warning related to the contraindication that does not disclose the contraindication.

20. The computer-readable medium of claim 15, wherein the processing device is further caused to:
receive a list of health providers that the patient permits receiving the notification of the contraindication.
